(19) United States
(12) Patent Application Publication
(10) Pub. No.: US 2006/0039935 A1
(43) Pub. Date: Feb. 23, 2006

(54) Composition of a transdermal delivery system, which modulates inflammation, via insitu systems, thereby promoting repair of injured, damaged or diseased joints, and soft tissue.

(75) Inventors: Stanley Charles Antosh, Palm Springs, CA (US); Anthony J. Meduri, New York City, NY (US)

Correspondence Address: OSMO, INC., 1271 VALDIVIA WAY, PALM SPRINGS, CA, 92262, US

(73) Assignees: Mr. Stanley Charles Antosh, Palm Springs, CA (US); Dr. Anthony J. Meduri, New York City, NY (US)

(21) Appl. No.: 10/711,078

(22) Filed: Aug. 20, 2004

(51) Int. Cl. A61K 31/455; A61K 31/4172; A61K 31/385; A61L 15/16

(52) U.S. Cl. 424/401; 424/448; 514/356; 514/440; 514/400

(57) ABSTRACT

The present invention relates to a composition comprising a mixture of nutritional co-factors synergistically combined and applied as a transdermal surfactant. Said surfactant has the ability to introduce locally, required nutrients that modulate chronic or acute inflammation, thereby promoting the systemic repair of joint and soft tissue damage, with concomitant relief of pain. Each of the ingredients serves as a co-synergist in a series of intertwined biochemical loops to substantiate each individual ingredient and to form a positive synergistic response operating in concert. This provides the insitu systems of the body nutrients required to repair injured, damaged or diseased joints, and soft tissue as a result of oxidative insult, stress, injury or disease.

### BACKGROUND OF INVENTION

Current U.S. Class: 514/310; 424/59; 424/61; 424/70.1; 424/400; 424/450; 424/489; 514/159; 514/256 International Class: A61K 031/47 Field of Search: 424/59,61,78,480,489,70,400 514/159,254,310

References Cited [Referenced By] U.S. Patent Documents: U.S. Pat. No. 4,968,685 1990-11 Grollier L'Oreal Composition for inducing and stimulating hair growth and retarding its loss, based on nicotinic esters and pyrimidine derivatives U.S. Pat. No. 5,133,958 1992-07 Stuckler Agent for nail, skin and hair care U.S. Pat. No. 5,215,759 1993-06 Mausner Chanel, Inc. Cosmetic composition U.S. Pat. No. 5,318,960 1994-06 Toppo Toppo; Frank System for transdermal delivery of pain relieving substances U.S. Pat. No. 5,954,675 Method of ultrasonic therapy 1999-09-21 U.S. Pat. No. 5,496,827 Compositions for the trans-dermal delivery of nutrients 1996-03-05 U.S. Pat. No. 5,451,407 Reduction or prevention of skin irritation or sensitization during transdermal administration of a irritating or sensitizing drug 1995-09-19 U.S. Pat. No. 6,398,753 Ultrasound enhancement of percutaneous drug absorption 2002-06-04 U.S. Pat. No. 6,316,490 Substituted aryl compounds useful as modulators of acetylcholine receptors 2001-11-13 U.S. Pat. No. 6,283,956 Reduction, elimination, or stimulation of hair growth 2001-09-04 U.S. Pat. No. 6,194,581 Substituted pyridines useful as modulators of acetylcholine receptors 2001-02-27 U.S. Pat. No. 6,030,374 Ultrasound enhancement of percutaneous drug absorption 2000-02-29 U.S. Pat. No. 5,985,860 System for transdermal delivery of pain relieving substances 1999-11-16 U.S. Pat. No. 5,723,477 Modulators of acetylcholine receptors 1998-03-03 U.S. Pat. No. 5,705,512 Modulators of acetylcholine receptors 1998-01-06 U.S. Pat. No. 5,703,100 Modulators of acetylcholine receptors 1997-12-30 U.S. Pat. No. 5,677,459 Methods for the preparation of modulators of acetylcholine receptors 1997-10-14 U.S. Pat. No. 5,594,011 Modulators of acetylcholine receptors 1997-01-14 U.S. Pat. No. 5,240,945 Method and compositions for treating acne 1993-08-31

### OTHER REFERENCES

Kikuchi K, Tagami H, Comparison of the effects of daily applications between topical corticosteroid and tacrolimus ointments on normal skin: evaluation with noninvasive methods. Dermatology. 2002; 205(4):378-82.
Baynes R E, Brooks J D, Mumtaz M, Riviere J E. Effect of chemical interactions in pentachlorophenol mixtures on skin and membrane transport. Toxicol Sci. 2002 October; 69(2):295-305.
Realdon N, Ragazzi E, Morpurgo M, Ragazzi E. of processing conditions in the manufacture of O/W creams: II. Effect on drug availability. Farmaco. 2002 May; 57(5):349-53.
Riviere J E, Qiao G, Baynes RE, Brooks JD, Mumtaz M. Mixture component effects on the in vitro dermal absorption of pentachlorophenol. Arch Toxicol. 2001 August; 75(6):329-34.
Bonina F P, Puglia C, Barbuzzi T, de Caprariis P, Palagiano F, Rimoli MG, Saija A. vitro and in vivo evaluation of polyoxyethylene esters as dermal prodrugs of ketoprofen, naproxen and diclofenac. Eur J Pharm Sci. 2001 September; 14(2): 123-34.
Issachar N, Gall Y, Borrel M T, Poelman M C. Correlation between percutaneous penetration of methyl nicotinate and sensitive skin, using laser Doppler imaging. Contact Dermatitis. 1998 October; 39(4):182-6.
Baynes R E, Monteiro-Riviere N A, Qiao G L, Riviere J E. Cutaneous toxicity of the benzidine dye direct red 28 applied as mechanistically-defined chemical mixtures (MDCM) in perfused porcine skin. Toxicol Lett. 1997 December; 93(2-3): 159-69.
Baynes R E, Brownie C, Freeman H, Riviere J E In vitro percutaneous absorption of benzidine in complex mechanistically defined chemical mixtures. Toxicol Appl Pharmacol. 1996 December; 141(2):497-506.
Williams P L, Thompson D, Qiao GL, Monteiro-Riviere N, Riviere J E. use of mechanistically defined chemical mixtures (MDCM) to assess mixture component effects on the percutaneous absorption and cutaneous disposition of topically exposed chemicals. II. Development of a general dermato-pharmacokinetic model for use in risk assessment. Toxicol Appl Pharmacol. 1996 December; 141(2):487-96.
Qiao GL, Brooks J D, Baynes R E, Monteiro-Riviere N A, Williams P L, Riviere J E. The use of mechanistically defined chemical mixtures (MDCM) to assess component effects on the percutaneous absorption and cutaneous disposition of topically exposed chemicals. I. Studies with parathion mixtures in isolated perfused porcine skin. Toxicol Appl Pharmacol. 1996 December; 141(2):473-86.
Realdon N, Ragazzi E, Dal Zotto M, Ragazzi E. Kinetics of release and simulated absorption of methyl nicotinate from different ointment formulations: in vitro-in vivo correlations. Pharmazie. 1996 February; 51(2): 113-6.
Leopold C S, Lippold BC. Enhancing effects of lipophilic vehicles on skin penetration of methyl nicotinate in vivo. J Pharm Sci. 1995 February; 84(2):195-8.
McElnay J C, Benson HA, Harland R, Hadgraft J. Phonophoresis of methyl nicotinate: a preliminary study to elucidate the mechanism of action. Pharm Res. 1993 December; 10(12): 1726-31.
Kompaore F, Tsuruta H. In vivo differences between Asian, black and white in the stratum corneum barrier function. Int Arch Occup Environ Health. 1993; 65(1 Suppl):S223-5.
Treffel P, Gabard B. Feasibility of measuring the bioavailability of topical ibuprofen in commercial formulations using drug content in epidermis and a methyl nicotinate skin inflammation assay. Skin Pharmacol. 1993; 6(4):268-75.
Leopold C S, Lippold B C. A new application chamber for skin penetration studies in vivo with liquid preparations. Pharm Res. 1992 September; 9(9):1215-8. No abstract available.
Roskos K V, Bircher A J, Maibach H I, Guy R H Pharmacodynamic measurements of methyl nicotinate percutaneous absorption: the effect of aging on microcirculation. Br J Dermatol. 1990 February; 122(2):165-71.
Poelman M C, Piot B, Guyon F, Deroni M, Leveque J L. Assessment of topical non-steroidal anti-inflammatory drugs. J Pharm Pharmacol. 1989 October; 41(10):720-2.
Walters K A, Walker M, Olejnik O Non-ionic surfactant effects on hairless mouse skin permeability characteristics. J Pharm Pharmacol. 1988 August; 40(8): 525-9.
Roskos K V, Guy RH, Maibach H I. Percutaneous absorption in the aged. Dermatol Clin. 1986 July; 4(3):455-65. Review.
Guy R H, Tur E, Bjerke S, Maibach H I. Are there age and racial differences to methyl nicotinate-induced vasodilatation in human skin?J Am Acad Dermatol. 1985 June; 12(6): 1001-6.
Osamura H, Jimbo Y, Ishihara M. Skin penetration of nicotinic acid, methyl nicotinate, and butyl nicotinate in the guinea pig—comparison of in vivo and excised skin, and the effects of four dermatologic conditions. J Dermatol. 1984 October; 11(5):471-81. No abstract available.
Tur E, Guy R H, Tur M, Maibach H I. Noninvasive assessment of local nicotinate harmacodynamics by photoplethysmography. J Invest Dermatol. 1983 June; 80(6):499-503.
Guy R H, Maibach H I. Rapid radial transport of methyl nicotinate in the dermis. Arch Dermatol Res. 1982; 273(1-2):91-5.
Albery W J, Hadgraft J Percutaneous absorption: in vivo experiments. J Pharm Pharmacol. 1979 March; 31(3):140-7.
Hadgraft J, Hadgraft J W, Sarkany I. Proceedings: The effect of thermodynamic activity on the percutaneous absorption of methyl nicotinate from water glycerol mixtures. J Pharm Pharmacol. 1973 December; 25:Suppl:122P-123. No abstract available.
Hadgraft J, Hadgraft J W, Sarkany I. The effect of glycerol on the percutaneous absorption of methyl nicotinate. Br J Dermatol. 1972 July; 87(1):30-6. No abstract available.
Fountain R B, Baker B S, Hadgraft J W, Sarkany I. The rate of absorption and duration of action of four different solutions of methyl nicotinate. Br J Dermatol. 1969 March; 81(3):202-6. No abstract available.
Reinberg A E, Soudant E, Koulbanis C, Bazin R, Nicolai A, Mechkouri M, Touitou Y. Circadian dosing time dependency in the forearm skin penetration of methyl and hexyl nicotinate. Life Sci. 1995; 57(16):1507-13.
McElnay J C, Benson H A, Harland R, Hadgraft J. Phonophoresis of methyl nicotinate: a preliminary study to elucidate the mechanism of action. Pharm Res. 1993 December; 10(12): 1726-31.
Kompaore F, Tsuruta H. In vivo differences between Asian, black and white in the stratum corneum barrier function. Int Arch Occup Environ Health. 1993; 65(1 Suppl):S223-5.
Lahti A, Kopola H, Harila A, Myllyla R, Hannuksela M Assessment of skin erythema by eye, laser Doppler flowmeter, spectroradiometer, two-channel erythema meter and Minolta chroma meter. Arch Dermatol Res. 1993; 285(5):278-82.
Kompaore F, Marty J P, Dupont C. In vivo evaluation of the stratum corneum barrier function in blacks, Caucasians and Asians with two noninvasive methods. Skin Pharmacol. 1993; 6(3):200-7.
Chan S Y, Li Wan Po A. Quantitative evaluation of drug-induced erythema by using a tristimulus colour analyzer: experimental design and data analysis. Skin Pharmacol. 1993; 6(4):298-312.
Treffel P, Gabard B. Feasibility of measuring the bioavailability of topical ibuprofen in commercial formulations using drug content in epidermis and a methyl nicotinate skin inflammation assay. Skin Pharmacol. 1993; 6(4):268-75.
Harvell J, Hussona-Saeed I, Maibach H I. Changes in transepidermal water loss and cutaneous blood flow during the menstrual cycle. Contact Dermatitis. 1992 November; 27(5):294-301.
Leopold C S, Lippold B C. A new application chamber for skin penetration studies in vivo with liquid Pharm Res. 1992 September; 9(9):1215-8. No abstract available.
Wahlberg E, Olofsson P, Swedenborg J, Fagrell B. Effects of local hyperemia and edema on the biological zero in laser Doppler fluxmetry (LD). Int J Microcirc Clin Exp. 1992 May; 11(2):157-65.
Morrow J D, Awad J A, Oates J A, Roberts L J 2nd. Identification of skin as a major site of prostaglandin D2 release following oral administration of niacin in humans. J Invest Dermatol. 1992 May; 98(5):812-5.
Duteil L, Queille C, Poncet M, Czernielewski J. Processing and statistical analysis of laser Doppler data applied to the assessment of systemic anti-inflammatory drugs. J Dermatol Sci. 1991 September; 2(5):376-82.
Elsner P, Maibach H I. Cutaneous responses to topical methyl nicotinate in human forearm and vulvar skin. J Dermatol Sci. 1991 September; 2(5):341-5.
Berardesca E, Cespa M, Farinelli N, Rabbiosi G, Maibach H. In vivo transcutaneous penetration of nicotinates and sensitive skin. Contact Dermatitis. 1991 July; 25(1):35-8.
Tur E, Maibach H I, Guy R H. Percutaneous penetration of methyl nicotinate at three anatomic sites: evidence for an appendageal contribution to transport? Skin Pharmacol. 1991; 4(4):230-4.
Duteil L, Queille C, Poncet M, Ortonne J P, Czernielewski J. Objective assessment of topical corticosteroids and non-steroidal anti-inflammatory drugs in methyl-nicotinate-induced skin inflammation. Clin Exp Dermatol. 1990 May; 15(3): 195-9.
Lanigan S W, Cotterill J A Reduced vasoactive responses in port wine stains. Br J Dermatol. 1990 May; 122(5):615-22.
Roskos K V, Bircher A J, Maibach H I, Guy R H Pharmacodynamic measurements of methyl nicotinate percutaneous absorption: the effect of aging on microcirculation. BrJ Dermatol. 1990 February; 122(2):165-71.
Berardesca E, Maibach H I Racial differences in pharmacodynamic response to nicotinates in vivo in human skin: black and white. Acta Derm Venereol. 1990; 70(1):63-6.
Larmi E, Lahti A, Hannuksela M. Effects of infra-red and neodymium yttrium aluminium garnet laser irradiation on non-immunologic immediate contact reactions to benzoic acid and methyl nicotinate. Derm Beruf Umwelt. 1989 November-December; 37(6):210-4.
Larmi E. PUVA treatment inhibits nonimmunologic immediate contact reactions to benzoic acid and methyl nicotinate. Int J Dermatol. 1989 November; 28(9):609-11.
Poelman M C, Piot B, Guyon F, Deroni M, Leveque J L. Assessment of topical non-steroidal anti-inflammatory drugs. J Pharm Pharmacol. 1989 October; 41(10):720-2.
Ylipieti S, Lahti A. Effect of the vehicle on non-immunologic immediate contact reactions. Contact Dermatitis. 1989 August; 21(2):105-6. No abstract available.
Kujala T, Lahti A Duration of inhibition of non-immunologic immediate contact reactions by acetylsalicylic acid. Contact Dermatitis. 1989 July; 21(1):60-1. No abstract available.
Gean C J, Tur E, Maibach H I, Guy R H. Cutaneous responses to topical methyl nicotinate in black, oriental, and caucasian subjects. Arch Dermatol Res. 1989; 281 (2):95-8.
Larmi E. Systemic effect of ultraviolet irradiation on non-immunologic immediate contact reactions to benzoic acid and methyl nicotinate. Acta Derm Venereol. 989; 69(4):296-301.
Fergusson D A. Systemic symptoms associated with a rubefacient. BMJ. 1988 November; 19; 297 (6659):1339. No abstract available.
Johansson J, Lahti A. Topical non-steroidal anti-inflammatory drugs inhibit non-immunologic immediate contact reactions. Contact Dermatitis. 1988 September; 19(3): 161-5.
Walters K A, Walker M, Olejnik O. Non-ionic surfactant effects on hairless mouse skin permeability characteristics. J Pharm Pharmacol. 1988 August; 40(8): 525-9.
Lahti A. Terfenadine does not inhibit non-immunologic contact urticaria. Contact Dermatitis. 1987 April; 16(4):220-3.
Lahti A, Vaananen A, Kokkonen E L, Hannuksela M. Acetyl-salicylic acid inhibits non-immunologic contact urticaria. Contact Dermatitis. 1987 March; 16(3):133-5.
Leahy D E, Wait Ark. Solute transport resistance at water-oil interfaces. J Pharm Sci. 1986 December; 75(12):1157-61.
Roskos K V, Guy R H, Maibach H I. Percutaneous absorption in the aged. Dermatol Clin. 1986 July; 4(3):455-65. Review.
Koch E M, Kohler C O. [Diurnal dependence of skin reactions in the administration of external substances]Z Hautkr. 1986 Mar. 15; 61(6):381-93. German.
Lahti A, McDonald DM, Tammi R, Maibach H I. Pharmacological studies on nonimmunologic contact urticaria in guinea pigs. Arch Dermatol Res. 1986; 279(1):44-9.
Lahti A, Maibach H I. Long refractory period after one application of nonimmunologic contact urticaria agents to the guinea pig ear. J Am Acad Dermatol. 1985 October; 13(4):585-9.
Wilkin J K, Fortner G, Reinhardt L A, Flowers O V, Kilpatrick S J, Streeter W C. Prostaglandins and nicotinate-provoked increase in cutaneous blood flow. Clin Pharmacol Ther. 1985 September; 38(3):273-7.
Tur E, Maibach H I, Guy RH. Spatial variability of vasodilatation in human forearm skin. Br J Dermatol. 1985 August; 113(2): 197-203.
Lahti A, Maibach H I Species specificity of non-immunologic contact urticaria: guinea pig, rat, and mouse. J Am Acad Dermatol. 1985 July; 13(1):66-9.
Guy R H, Tur E, Bjerke S, Maibach H I Are there age and racial differences to methyl nicotinate-induced vasodilatation in human skin? J Am Acad Dermatol. 1985 June; 12(6): 1001-6.
Guy R H, Hadgraft J, Maibach H I Percutaneous absorption in man: a kinetic approach. Toxicol Appl Pharmacol. 1985 March; 30; 78(1):123-9.
Lahti A, Maibach H I An animal model for non-immunologic contact urticaria. Toxicol Appl Pharmacol. 1984 November; 76(2):219-24.
Osamura H, Jimbo Y, Ishihara M. Skin penetration of nicotinic acid, methyl nicotinate, and butyl nicotinate in the guinea pig—comparison of in vivo and excised skin, and the effects of four dermatologic conditions. J Dermatol. 1984 October; 11(5):471-81. No abstract available.
Guy R H, Wester R C, Tur E, Maibach H I Noninvasive assessments of the percutaneous absorption of methyl nicotinate in humans. J Pharm Sci. 1983 September; 72(9):1077-9.
Tur E, Guy R H, Tur M, Maibach Him Noninvasive assessment of local nicotinate harmacodynamics by photoplethysmography. J Invest Dermatol. 1983 June; 80(6):499-503.
Fleming R, Guy RH, Hadgraft J. Kinetics and thermodynamics of interfacial transfer. J Pharm Sci. 1983 February; 72(2): 142-5.
Guy R H, Maibach H I. Rapid radial transport of methyl nicotinate in the dermis. Arch Dermatol Res. 1982; 273(1-2):91-5.
Matushima I, Osamura H. [Studies on the permeability of human skin to nicotinic acid, methyl nicotinate and butyl nicotinate.—with emphasis on the influence of liquid vehicles and that of water immersion (author's transl)] Nippon Hifuka Gakkai Zasshi. 1980 July; 90(8):687-99. Japanese. No abstract available.
Bunting J W, Laderoute K R, Norris D J The pH dependence of xanthine oxidase catalysis in basic solution. Can J Biochem. 1980 May; 58(5):394-8.
Albery W J, Hadgraft J. Percutaneous absorption: in vivo experiments. J Pharm Pharmacol. 1979 March; 31(3):140-7.
Hadgraft J, Hadgraft J W, Sarkany I. Proceedings: The effect of thermodynamic activity on the percutaneous absorption of methyl nicotinate from water glycerol mixtures. J Pharm Pharmacol. 1973 December; 25:Suppl:122P-123. No abstract available.
Hadgraft J, Hadgraft J W, Sarkany I. The effect of glycerol on the percutaneous absorption of methyl nicotinate. Br J Dermatol. 1972 July; 87(1):30-6. No abstract available.
Fountain RB, Baker B S, Hadgraft J W, Sarkany I. The rate of absorption and duration of action of four different solutions of methyl nicotinate. Br J Dermatol. 1969 March; 81(3):202-6. No abstract available.
van der Wegen P A. [Crystal reactions in methyl nicotinate]Pharm Weekbl. 1966 Jan. 14; 101(2):33-6. Dutch. No abstract available.
Tonder J E, Olesen P H. Agonists at the alpha4beta2 nicotinic acetylcholine receptors: structure-activity relationships and molecular modelling. Curr Med Chem. 2001 May; 8(6):651-74. Review.
Narahashi T, Fenster C P, Quick M W, Lester R A, Marszalec W, Aistrup G L, Sattelle D B, Martin BR, Levin ED. Symposium overview: mechanism of action of nicotine on neuronal acetylcholine receptors, from molecule to behavior. Toxicol Sci. 2000 October; 57(2):193-202.
Yamamoto I, Tomizawa M, Saito T, Miyamoto T, Walcott EC, Sumikawa K. Structural factors contributing to insecticidal and selective actions of neonicotinoids. Arch Insect Biochem Physiol. 1998; 37(1):24-32.
Kanne D B, Abood L G. Synthesis and biological characterization of pyridohomotropanes. Structure-activity relationships of conformationally restricted nicotinoids. J Med Chem. 1988 March; 31(3):506-9.
Yamamoto I Nicotinoids as insecticides. Adv Pest Control Res. 1965; 6: 231-60. Review. No abstract available.
Karjalainen A, Heikkinen J, Savolainen M J, Backstrom A C, Kesaniemi Y A. Mechanisms regulating LDL metabolism in subjects on peroral and transdermal estrogen replacement therapy. Arterioscler Thromb Vasc Biol. 2000 April; 20(4): 1101-6.
Green J T, Rhodes J, Thomas G A, Evans B K, Feyerabend C, Russell M A, Sandborn W J Nicotine carbomer enemas-pharmacokinetics of a revised formulation. Ital J Gastroenterol Hepatol. 1998 June; 30(3):260-5.
Pierard GE, Van Cromphaut I, Pierard-Franchimont C, Gaspard U, Kulbertus H. [The skin and transdermal applications] Rev Med Liege. 1997 September; 52(9):585-8. Review. French. No abstract available.
Mahmood I Clinical pharmacokinetics and pharmacodynamics of selegiline. An update. Clin Pharmacokinet. 1997 August; 33(2):91-102. Review.
Sips A J, Netelenbos J C, Barto R, Verburgh C A, Nieboer C, van der Vijgh W J. Topically applied low-dose calcitriol has no calciotropic effect in patients with stable plaque psoriasis. J Am Acad Dermatol. 1994 June; 30(6):966-9.
Morimoto K, Morisaka K. Related Articles, [Theory and practice for design of transdermal and transmucosal drug delivery systems] Nippon Rinsho. 1989 June; 47(6): 1255-60. Japanese. No abstract available.
Lobo R A. Absorption and metabolic effects of different types of estrogens and progestogens. Obstet Gynecol Clin North Am. 1987 March; 14(1):143-67. Review.
Schultz T W, Ando H Y. A relationship between diffusional transport in lipid membranes and a lipophilic-eutectic parameter. J Theor Biol. 1986 Dec. 7; 123(3):367-375.

### Field of the Invention

The present invention relates to the field of nutritional biochemistry and further to a trans-dermal method for delivery of nutrients. This provides the body with required nutrients, which thereby enable the insitu systems to modulate inflammation, thus repair injury, damaged or diseased joints, and soft tissue as a result of oxidative insult, stress, injury or disease.

### Background of the Invention

Inflammation is a reaction of living tissues to injury. The discipline of pathology makes a fundamental distinction between acute and chronic inflammation. Acute inflammation comprises the immediate and early response to an injurious agent and is basically a defensive response that paves the way for repair of the damaged site. Chronic inflammation results from stimuli that are persistent.

Arachidonic acid plays a central role in a biological control system where such oxygenated derivatives as prostaglandins, thromboxanes, and leukotrienes are mediators. The leukotrienes are formed by transformation of arachidonic acid into an unstable epoxide intermediate, leukotriene A4, which can be converted enzymatically by hydration to leukotriene B4, and by addition of glutathione to leukotriene C4. This last compound is metabolized to leukotrienes D4 and E4 by successive elimination of a gamma-glutamyl residue and glycine. Slow-reacting substance of anaphylaxis consists of leukotrienes C4, D4, and E4. The cysteinyl-containing leukotrienes are potent bronchoconstrictors, increase vascular permeability in postcapillary venules, and stimulate mucus secretion. Leukotriene B4 causes adhesion and chemotactic movement of leukocytes and stimulates aggregation, enzyme release, and generation of superoxide in neutrophils. Leukotrienes C4, D4, and E4, which are released from the lung tissue of asthmatic subjects exposed to specific allergens, seem to play a pathophysiological role in immediate hypersensitivity reactions. These leukotrienes, as well as leukotriene B4, have pro-inflammatory effects. Leukotriene B(4) (LTB(4)) is a potent, proinflammatory mediator involved in the pathogenesis of a number of diseases including psoriasis and rheumatoid arthritis.

Proprietary formulations of the amino acid L-histidine are under development as pharmaceutical agents because of the molecule's antioxidant and anti-inflammatory properties. L-histidine has been well characterized in terms of probable dietary requirements, plasma and tissue concentrations, pharmacokinetics, metabolism and excretion, and medical conditions related to physiologic handling. Previous experience with histidine dosing in the literature is extensive, and both clinical and preclinical data suggest that histidine administration is very safe. L-histidine has been shown to scavenge both the hydroxyl radical and singlet oxygen (102) in many studies. These interactions may involve free histidine, small histidine-containing peptides such as carnosine, and histidine residues in proteins. Histidine appears to interfere with redox reactions involving iron and perhaps other metal ions and to interact directly with 102; the ability of histidine to scavenge 102, a toxic oxygen species of increasing concern, has been well established in the laboratory. Many recent studies have demonstrated the therapeutic efficacy of “pharmacologic” doses of L-histidine in animal models of inflammatory conditions, particularly gastrointestinal conditions and cardiac ischemia-reperfusion injury, and have specifically linked the anti-inflammatory capabilities of histidine to its ability to scavenge toxic oxygen species. The maintenance of histidine pools, therefore, may contribute to the body's physiologic antioxidant capacity. Taken together, the data suggest that histidine supplementation could provide a safe, efficacious method to increase antioxidant protection.

Endogenous L-carnosine is a di-peptide, synthesized in the mammalian tissue and brain by an enzyme, carnosine-synthetase, which bonds the amino acids beta-alanine and histidine. The enzyme carnosinase, maintains carnosine equilibrium (inactivates) by cleaving the peptide bond in the plasma. This yields the amino acids beta-alanine and histidine. The half-life of carnosine in plasma is about one minute. The only mammals that maintain plasma carnosinase are humans, primates and the Syrian Golden Hamster.

Carnosine seems to be concentrated in actively contracting muscles. In patients with degenerative disease, for example MS or muscular dystrophy, carnosine levels are lower.

The concentration of carnosine in muscles also appears to correlate with age. Older people have lower levels of carnosine in their muscles. Carnosine is an anti-aging product because of its effects on advanced glycosylation end-products or AGE's. AGE's are abnormal, cross-linked and oxidized proteins that play a role in the aging process. Carnosine appears to block the formation of AGE's and ensures the proper formation of proteins on the DNA level. For this reason there is great interest in using carnosine for the complications of diabetes, such as cataracts, neuropathy and kidney failure, which all arise from glycosylation.

Carnosine has anti-carbonylation qualities, which prevents the age-related degradation of the body's proteins. Carnosine is a potent antioxidant, which effectively quenches the most destructive of the free radicals—the hydroxyl radical and the peroxyl radical.

Carnosine also has a remarkable ability to rejuvenate cells approaching senescence—restoring normal appearance and extending cellular lifespan. As a wound healing agent carnosine has the ability to rejuvenate connective tissue. Additionally it protects the brain from plaque formation that leads to senility or Alzheimers disease. Carnosine enables the heart muscle to contract more effectively through enhancement of calcium response in heart myocytes. It also protects cellular DNA from oxidative damage, which accumulates with age. Finally, carnosine helps prevent skin collagen cross-linking which leads to loss of elasticity, wrinkles, macro-molecular disorganization and loss of extra-cellular matrix. Carnosine extends the functional life of the body's key building blocks, cells, proteins, DNA, lipids and is an agent of longevity. Besides reversing the signs of aging in cells approaching senescence and increasing cellular life span, studies show that carnosine is effective against all forms of protein modification. Carnosine levels decline as we age. Muscle levels drastically decline over 63% from ages 10 to 70, which accounts for the normal age related decline in muscle mass and function.

Carcinine, also a di-peptide (resistant to hydrolysis by carnosinase) is synthesized from beta alanine and histamine. First, histidine is metabolized by histidine decarboxylase (not found in the eyes) into histamine. Carcinine synthetase (an abundant, stable enzyme) bonds beta alanine and histamine to produce carcinine. The seat of carcinine synthesis is the CNS (Central Nervous System) where carcinine synthetase is 15 times higher than elsewhere. Carcinine is an antioxidant di-peptide that specifically addresses CNS oxidation. Additionally, carcinine a catabolic metabolite of histamine accumulates in the heart after completing its function in the CNS thereby increasing cardiac output.

With beta alanine, L-histidine and its metabolite histamine introduced locally, at the site of affliction, it is logical to assume that these tissues and cells will take up these molecules and synthesis carnosine and/or carcinine.

### SUMMARY OF INVENTION

The present invention pertains to a medicating composition for dermal application comprising methyl nicotinate, nicotinic acid, alpha lipoic acid, beta alanine, histidine, carnosine, copper, pyridoxal-5-phosphate, medium chain triglycerides, phosphatidyl choline, glycerol, polysorbate 80, propylene glycol, and water. This composition of nutrient co-factors is applied locally via a trans-dermal method. This provides the insitu system's of the body with required nutrients to control inflammation and repair injured, damaged or diseased joints, and soft tissue as a result of oxidative insult, stress, injury or disease. Each of the ingredients serves as a co-synergist in a series of intertwined biochemical loops to substantiate each individual ingredient and to form a positive synergistic response operating in concert.

Typical dosage/usage will depend on factors such as size, age, health of the user, and nature of the inflammation, along with location and duration of the injury/event. This treatment is effective when administered on a chronic or acute basis contingent on the duration, severity and physical rehabilitation of the injury/affliction.

### DETAILED DESCRIPTION

Methyl nicotinate is present for its ability to easily and consistently pass through the epidermis and as such carry with it nutrients to the target area, there-by by passing the G. I. tract and liver. This eliminates the usual degradation and metabolism associated with oral ingestion. Nicotinate increases the surface temperature of the skin (warming) and causes a significant release of prostaglandins (PGE 2) from the skin, as well as stimulating histamine release from the of the mast of the cells in the tissue thereby initiating the autacoid response of the specific immune system. This facilitates blood circulation to and then away from the site of an injury. Increased circulation facilitates the repair process by supplying needed molecules and removing deleterious ones. Methyl nicotinate also interacts with other members of the nicotinoid super-family and produces a catalyzing effect on the absorption of nutrients and expeditious transport of nutrients to deep soft tissues and joints.

Nicotinic acid is used as a complement to methyl nicotinate. Nicotinic acid is used topically to act as an additional electron donor from which to draw upon for heightened tissue repair. Nicotinate forms its own biochemical loop interacting with the epidermis and creating a “skin-flush”, as well as, co-synergizing with P-5-P, methyl nicotinate, and copper.

Nicotinic acid receptors know as the G-Protein-Coupled Receptor G(1) are highly expressed in adipose tissue. By including both methyl nicotinate and nicotinic acid in this formula there is a 2 pronged “time released” effect on G(1). This serves to stimulate the release of fatty acids from the adipose tissue. Adipose tissue contains (stores) various fatty acids—one of which is arachadonic acid (AA). AA is the main constituent of the autacoid—prostaglandins, part of the specific immune system which initiates the repair process. The combination of these nicotinoids provides the formulation with the beginning of a healing loop to undo the internal oxidative insult due to injury or atrophy.

Pyridoxol-5-Phosphate is a potent and active form of Vitamin B-6. P-5-P is a vital cofactor—necessary in the methylation process, which is endemic in the biochemical process called “Life”. By directly introducing P-5-P, where it is needed—through a trans-dermal system we have eliminated the associated degradation resulting from oral ingestion and the concomitant pass through the GI tract and liver. P-5-P can work as a catalyst with the requiste enzymes, amino acids and other nutrients/vitamins to produce a reaction(s).

P-5-P is required for the absorption and movement (transport) into the bloodstream and tissue of amino acids—as such it is a conduit provider. P-5-P forms its own facilitator loop allowing for greater biochemical uptake of copper, as well as, the amino acids beta alanine and L-histidine. Of other importance, P-5-P (as an anti-oxidant) minimizes “free” copper from oxidizing HDL cholesterol more effectively than vitamin E. It helps to ensure copper's utility in the formulation and increases its transport and bio-availability. Thus, P-5-P forms a conduit loop, an antioxidant loop, a methylation loop while catalyzing histidine into histamine and further into carcinine.

Alpha-Lipoic Acid—as a thiol ALA is a universal antioxidant, which interacts with other water and fat-soluble antioxidants. ALA potentiates them and increases their respective bio-absorbability by serving to re-cycle these anti-oxidants. forms an exceptionally strong antioxidant loop while interacting with -5-P, copper, histidine, beta alanine, methyl nicotinate and nicotinic acid substantiating the respective antioxidant benefits of those nutrients and acting as a biochemical overseer for anti-oxidation and free radical quenching.

ALA also works along with the nicotinates to seamlessly pass through the skin barrier and protect and buffer any and all metabolic by-products deposited beyond the plasma membrane; in this way it serves both as an antioxidant loop coordinator and becomes a vital part of the intra-epidermal transport loop. ALA serves as a prosthetic group, a scaffold of the H-protein of the glycine cleavage system and the dihydrolipoamide acyltransferases (E2) of the pyruvate, alpha ketoglutarate and branched-chain alpha-keto acid dehydrogenase complexes. ALA and its reduced form, di-hydrolipoic acid scavenges ROS (reactive oxygen species). ALA has proven beneficial clinical effects on oxidative stress models by blocking the reuptake/reactivation of the neuro-toxic glutamate metabolites.

Copper—A ubiquitous mineral, copper, is utilized by the body in a variety of ways. When added to this present composition copper provides an analgesic-like effect to the structures deep within the joint and soft tissues. Copper provides the biochemical infrastructure and scaffolding for the universal anti-oxidant, one of the body's principal free radical scavengers, superoxide dismutase (Cu SOD).

Copper is an element necessary for oxidation and absorption of iron and vitamin C (ascorbic acid) in digestion. Copper also acts as a catalyst in the formation of hemoglobin, the oxygen-carrying blood component, and a condition similar to iron-deficiency (anemia) has been produced experimentally in cases of copper deficiency. Thus, copper serves as an antioxidant loop, part of the overall healing loop and a part of the pain diminishment loop. Working in synergy with P-5-P free copper is made safe, non-toxic and bioavailable to become SOD—countering oxidative tissue and joint insult.

L-Histidine forms its own loop with respect to entry. As a potent anti-oxidant histidine is also a pro-stimulator of circulation when topically applied. But, much more as a co-synergist with several other substrates to form a histidine -histamine-carcinine-carnosine loop, protecting tissues from further oxidative stress/damage while providing a readily available scaffold for neural-transmission and pain mitigation. Histidine acts as a neuro-modulator, secondary neurotransmitter, neuro-protectant against hydroxyl radicals, a cellular homeostasis regulator and anti-inflammatory agent.

The carnosine loop elucidated is such. Histidine in the presence of beta alanine, ATP, and carnosine synthetase is metabolized to yield carnosine. Carnosine is a biologically super-active peptide, with a multitude of functions. Carnosine synthetase is found in tissue both slow twitch and fast twitch. This is where carnosine is synthesized.

This is also where carnosine effects are most required. This method of trans-dermal, local introduction into tissue cells—histidine and beta alanine—tissue that contains carnosine synthetase, for the expressed, expedited purpose of synthesizing carnosine—where it is most needed—is indeed novel.

The metabolism of carnosine loop elucidated is such. Carnosine, a peptide is metabolized by carnosinase in the plasma. Yielding histidine and beta alanine which may then again be synthesized back into carnosine, however carnosine synthetase is needed, this enzyme is an enzyme that is under-abundant and unstable. This fact is expressed to limit the availability and quantity of carnosine.

An alternative metabolic process for histidine is into histamine. Histidine in the presence of histidine decarboxylase—with the required cofactor P-5-P is metabolized into histamine. Histamine, in its own right is a super-potent anti oxidant, anti-inflammatory, neuro modulator, neuro-protectant, neuro-transmitter amino acid. Like its sister molecule histidine, histamine has a multitude of biological functions. When histamine is present it is able to exert its activity as histamine, or it can be metabolized into carcinine.

Carcinine is synthesized in the presence of carcinine synthetase, histamine, beta alanine, ATP and the required co-factor P-5-P. Carcinine a potent anti oxidant is primarily active in the CNS (central nervous system). It has been shown that carcinine synthetase enzyme levels are 15 times higher in the CNS than elsewhere. The seat of carcinine synthesis and activity is the CNS.

After carcinine has exerted its activity as an anti oxidant in the CNS it is transported away in the plasma resulting in a smooth-muscle relaxatory effect, which lowers blood pressure. Additionally, carcinine as an anti oxidant accumulates in the heart expressing its activity there. Carcinine can be metabolized back into histamine and beta alanine, also by carnosinase, however, it is relatively resistant to its effects. Histamine and beta alanine are both oxidized into acetic acid. Additionally histamine could remain as histamine retaken up in the carcinine loop, or methylated into 3 methylhistamine.

The vast majority of histamine is methylated then oxidized to imidazole acetic acid, a molecule that can occupy the glycine receptor—preventing glutamate activity. Glutamate, an excitatory amino acid responsible for the “Pain Signal” cannot send the pain signal unless glycine occupies its receptor at the same time.

Histidine as you can understand, is therefore involved in its own neuro-hormonal loop, a synergized breakdown—conversion loop and an anti-inflammatory loop. Together with P-5-P and the nicotinoids, histidine forms a powerful inflammatory cascade and resulting/subsequent anti-inflammatory loop. By engendering the formation of site specific prostaglandins—tissue and joint insult is rapidly mitigated and systemic healing/replacement is permitted to occur at a much more rapid rate.

Beta alanine—the predominate branch amino acid in mammals and also the only naturally occurring branch amino acid is part of its own loop, the regenerating keratin loop—a constituent of the major molecular ingredient of human skin—keratin. Additionally, it is known to stimulate collagen (constituent of cartilage and platelets) and nucleic acid synthesis.

Beta alanine is an essential co-factor for the carcinine-carnosine loop—efficacious as an anti-oxidant and for the cellular regulation/repair process. Beta alanine together with histidine/histamine forms the carnosine-carcinine covalent bond. Beta alanine is a key molecule necessary for production of carcinine—found almost exclusively in the central nervous system (CNS) and acts as an arbiter and anti-inflammation regulator, antioxidant and neuro-modulation agent.

Beta alanine is also a key molecule necessary for production of carnosine, a vital part of this multi-clustered loop. Carnosine acts as a very powerful wound-healing agent, exhibiting anti-senescent effect at the cellular level. As well as, a potent time-dependent regulator of the intracellular “biological-clock” cascade, also serving as an autoimmune function protector.

Beta alanine additionally is a necessary component for the synthesis (non-vertebrates) of pantothenic acid (B5), which is a cofactor needed for the synthesis of Co A. Co A, a very interesting enzyme, initiates the Krebs cycle. The Krebs cycle is the system in the body that produces ATP (adenosine tri phosphate) energy. This cycle of energy is the most basic molecular production of energy in all cells of the body. Interestingly, injury and exercise deplete cells of ATP. ATP depleted cells, “pre-loaded” with beta alanine stimulates the uptake of beta alanine, and thus the “Krebs Cycle” and repair process.

Beta alanine, completely desensitizes the glycine receptor and then metabolizes into acetic acid—a molecule which is also able to occupy the glycine receptor. This receptor occupation prevents the neuron from sending a glutamate-induced signal a signal that results in feeling pain. With the glycine receptor occupied by acetic acid, the metabolite of beta alanine, a concomitant cessation of glutamate excitation is prevented. This prevention minimizes NMDA neuro-toxicity, a metabolite of glutamate excitation, allowing for a quick, utilizable uptake of the formulation substrates.

Leukotrienes B4 are the predominant pro-inflammatory molecule in the body. Leukotrienes B4, metabolites of arachadonic acid are rapidly, potently suppressed by beta alanine. Hence, this method of delivery of beta alanine and its ability to suppress leukotriene initiated inflammation is indeed novel.

Medium Chain Triglycerides—are involved in the surface absorption of the formulation and play a vital role in the ability of this formulation to pass thorough the skin's plasma membrane and also act as a carrier, bringing nutrients to injured joints and tissues. They improve and enhance the trans-dermal delivery of nutrients and as such are involved in the transport loop of the formulation.

Propylene Glycol and Polysorbate 80—are both non-ionic solvents, which serve as coupling agents in the formulation. They are both used principally in the transport loop. Both provide for the marriage and transport of water, nutrients and oils (MCT's) by acting as a bridge and conduit between the glycerol backbone of the MCT's and the water. Thus, permitting the oil, water, and nutrient mixture to restructure its molecular makeup and become one, while passing through the epidermis and dermis into the tissue, joint, cells, ultimately into the plasma. While allowing the nutrients as well as the metabolites to be become systemic, thereby allowing the required environment to be created for the desired healing/repairing processes.

Glycerol—is hydroscopic and acts as a moisturizing agent, more directly as a method to open this solutions' molecules, enabling better, more complete uptake through the pores of the skin, which allows better reception of the formulations substrates. Thus, glycerol is part of the transport and initial delivery loop. It provides the first entry into the skin barrier where propylene glycol and polysorbate-80, as well as, MCT's take over to provide a secondary transport and delivery system.

A transdermal healing composition has been described with reference to a particular embodiment. For one skilled in the art, other modifications and enhancements can be made without departing from the spirit and scope of the aforementioned claims.

Whilst endeavoring in the foregoing specification to draw attention to those features of the invention believed to be of particular importance it should be understood that the Applicant claims protection in respect of any patentable feature hereinbefore referred to whether or not particular emphasis has been placed thereon.

1. We claim a trans-dermal delivery composition comprising, in a liquid, between 0.01% and 1% methyl nicotinate by weight and between 0.01% and 5% by weight of vitamins, minerals and amino acids, selected from the group consisting of: pyridoxal-5-phosphate, nicotinic acid, copper, 3-aminopropionic acid (beta alanine), histidine, alpha lipoic acid, carnosine.

2. We claim a composition according to claim 1 further comprising between 1% and 50%, by weight in said liquid selected from the group consisting of medium chain triglycerides, phosphatidyl choline, glycerol, polysorbate 80, propylene glycol.

3. We claim a composition according to claim 1 wherein said liquid is comprised of water.

4. We claim a composition according to claim 3 wherein said liquid is further comprised of alcohol.

5. We claim a skin application substance comprising, in a liquid, between 0.01% and 1% methyl nicotinate by weight and between 0.01% and 5% by weight of vitamins, minerals and amino acids, selected from the group consisting of: pyridoxal-5-phosphate, nicotinic acid, copper, 3-aminopropionic acid (beta alanine), histidine, alpha lipoic acid, carnosine.

6. We claim a skin application substance according to claim 5 further comprising between 1% and 50%, by weight in said liquid selected from the group consisting of medium chain triglycerides, phosphatidy choline, glycerol, polysorbate 80, propylene glycol.

7. We claim a skin application substance according to claim 5 wherein said liquid is comprised of water.

8. We claim a skin application substance according to claim 7 wherein said liquid is further comprised of alcohol.

9. We claim a medicating composition for dermal application comprising, in a liquid, between 0.01% and 1% methyl nicotinate by weight and between 0.01% and 5% by weight of vitamins, minerals and amino acids, selected from the group consisting of: pyridoxal-5-phosphate, nicotinic acid, copper, 3-aminopropionic acid (beta alanine), histidine, alpha lipoic acid, carnosine.

10. We claim a medicating composition for dermal application according to claim 9 further comprising between 1% and 50%, by weight in said liquid selected from the group consisting of medium chain triglycerides, phosphatidy choline, glycerol, polysorbate 80, propylene glycol.

11. We claim a medicating composition for dermal application according to claim 9 wherein said liquid is comprised of water.

12. We claim a medicating composition for dermal application according to claim 11 wherein said liquid is further comprised of alcohol.
